(19) United States
(12) Patent Application Publication
(10) Pub. No.: US 2022/0005567 A1
(43) Pub. Date: Jan. 6, 2022

(54) Current Health Status Certification

(71) Applicant: Rememdia LC, Salt Lake City, UT (US)

(72) Inventor: Fraser M. Smith, Salt Lake City, UT (US)

(21) Appl. No.: 16/920,368

(22) Filed: Jul. 2, 2020

(51) Int. Cl. G16H 15/00; G16H 40/67; G16H 10/65; G16H 50/30; G16H 50/70; G16H 50/20; G06Q 50/26; G06F 21/60; H04L 9/06; G06F 21/62; G08B 5/22; H04L 29/08

(52) U.S. Cl. CPC G16H 15/00; G16H 40/67; G16H 10/65; G16H 50/30; G16H 50/70; G16H 50/20; H04L 2209/38; G06F 21/602; H04L 9/0637; G06F 21/6245; G08B 5/22; H04L 67/12; H04L 67/22; G06Q 50/265

(57) ABSTRACT

A technology is described for providing a determination regarding a person's health while maintaining privacy. In one example, health metrics for a person can be securely collected from sensor devices in communication with a mobile computing device during a baseline time period and baseline health metrics and a baseline health status can be defined using the one or more health metrics. Thereafter, a request for a current health status for the person can be received and current health metrics can be obtained for the person from sensor devices in communication with the mobile computing device. The current health metrics can be compared to the baseline health metrics to define the current health status for the person, and a certification message can be generated to be provided to a third-party certifying the current health status for the person.

### FIELD OF THE TECHNOLOGY

The present technology relates to improved systems, methods, and devices for estimating a health status of an individual person. More particularly, the present technology relates to systems, methods, and devices for estimating a current health status of a person by comparing current health metrics to baseline health metrics and generating a certification message certifying the current health status for the person, which may be provided to a third-party, such as for point of access admittance.

### BACKGROUND OF THE TECHNOLOGY

The risks associated with contracting and disseminating an infectious disease through public contact have posed challenges to developing public policy for commercial locales, group gatherings and confined travel. The difficulty of assessing the health state of a person who is asymptomatic, exhibits mild symptoms, or masks symptoms has led to public policy that can be prohibitive and can have detrimental effects on the economy and individual mental health. As an example, because of the potential of an infected person to communicate an infectious disease to others, public policy may prohibit gatherings of persons, limit gatherings to a limited number of persons, or prohibit or limit access of persons to certain commercial locales, such as restaurants, retail and other stores, entertainment venues, and others. As another example, due to a public fear of contracting an infectious disease, persons may avoid confined travel (e.g., air travel, metros, trains, busses, taxies, etc.). Determining a health status of a subject often requires a diagnosis by a medical practitioner during a physical or virtual office visit. However, the health status of a person may be detected without the assistance of a medical practitioner to place the person on notice that public contact or interaction should be avoided and the person should not enter public locations where the person could transmit an infectious disease.

### SUMMARY OF THE INVENTION

In light of the problems and deficiencies inherent in the prior art, disclosed herein are systems, methods, and devices configured to monitor indicators of health for a person and use those indicators to determine a current health state of the person and generate a health certification message indicating the current health state. Such a certification message can be conveyed to a third-party, such as to enable the third-party to permit or deny point of access admittance of the person, or in other words, admittance of the person to a locale, area, activity, object, etc. overseen by the third-party. In one example discussed herein, a method for providing a determination regarding a person's health while maintaining privacy, can include collecting, at a mobile computing device, one or more health metrics for a person from sensor devices in communication with the mobile computing device during a baseline time period. The method can further include the operation of defining baseline health metrics and a baseline health status using the one or more health metrics. A request for a current health status for the person can be received. The method can further comprise obtaining one or more current health metrics for the person from the mobile computing device, or the sensor devices in communication with the mobile computing device, wherein the current health metrics are obtained during a current time period. The method may further comprise comparing the current health metrics to the baseline health metrics to define the current health status for the person. And the method can further comprise generating a certification message at the mobile computing device. In one aspect, the certification message can be provided to a third-party to facilitate a determination by the third-party whether or not the person should be granted point of access admittance overseen by the third-party. In one example, a photograph of the person can be displayed with the certification message. The photograph can provide verification of the identity of the person.

In one aspect of the technology, a mobile computing device can provide a determination regarding a person's health while maintaining privacy. The mobile computing device can comprise at least one processor, and a plurality of sensor devices to monitor a person's medical metrics, wherein the plurality of sensor devices are in communication with the mobile computing device. The mobile computing device may include at least one memory device having a data store to store a plurality of data and instructions that, when executed, cause the mobile computing device to collect one or more health metrics for a person from the plurality of sensor devices in communication with the mobile computing device during a baseline time period. The instructions, when executed, cause the mobile computing device to further define baseline health metrics and a baseline health status using the one or more health metrics. The instructions, when executed, cause the mobile computing device to further receive a request for a current health status for the person. The mobile computing device can further receive current health metrics for the person from the plurality of sensor devices in communication with the mobile computing device, and the current health metrics are obtained during a current time period. In addition, the mobile computing device can further compare the current health metrics to the baseline health metrics to define a current health status for the person. The mobile computing device can display a certification message certifying the person's current health status on a screen of the mobile computing device. In one aspect, the certification message can be provided to a third-party, such as to facilitate a determination by the third-party whether or not the person should be granted point of access admittance.

In one aspect of the technology, a non-transitory machine readable storage medium having instructions embodied thereon, the instructions when executed by one or more processors, cause the one or more processors to perform a process comprising receiving one or more health metrics for a person from a plurality of sensor devices in communication with a mobile computing device during a baseline time period. The process further comprises defining baseline health metrics and a baseline health status using the one or more health metrics. The process further can include the operation of receiving a request for a current health status for the person. Current health metrics for the person from the plurality of sensor devices in communication with the mobile computing device can be received, and the current health metrics are obtained during a current time period. The process further includes comparing the current health metrics to the baseline health metrics to define a current health status for the person. In addition, a certification message certifying the current health status of the person may be displayed on a screen of the mobile computing device to enable the certification message to be provided to a third-party.

### BRIEF DESCRIPTION OF THE DRAWINGS

The present technology will become more fully apparent from the following description and appended claims, taken in conjunction with the accompanying drawings. Understanding that these drawings merely depict exemplary aspects of the present technology they are, therefore, not to be considered limiting of its scope. It will be readily appreciated that the components of the present technology, as generally described and illustrated in the figures herein, could be arranged and designed in a wide variety of different configurations. Nonetheless, the technology will be described and explained with additional specificity and detail through the use of the accompanying drawings in which:

FIG. 1 is a diagram illustrating aspects of the present technology for providing a determination regarding a person's health while maintaining privacy.

FIG. 2 is a block diagram that illustrates a system environment that includes a mobile computing device used in accordance with an example of the present technology.

FIG. 3 is a diagram illustrating another system environment that includes a third-party mobile computing device used in accordance with an example of the present technology.

FIG. 4 is a diagram that illustrates another system environment that includes a health check station used in accordance with an example of the present technology.

FIG. 5 is a flow diagram illustrating an example method for providing a current health status of a person.

FIG. 6 is block diagram illustrating an example of a computing device that may be used to execute a method for determining a current health status for a person.

### DETAILED DESCRIPTION OF EXEMPLARY ASPECTS OF THE TECHNOLOGY

The following detailed description of exemplary aspects of the technology makes reference to the accompanying drawings, which form a part hereof and in which are shown, by way of illustration, exemplary aspects in which the technology can be practiced. While these exemplary aspects are described in sufficient detail to enable those skilled in the art to practice the technology, it should be understood that other aspects can be realized and that various changes to the technology can be made without departing from the spirit and scope of the present technology. Thus, the following more detailed description of the aspects of the present technology is not intended to limit the scope of the technology, as claimed, but is presented for purposes of illustration only and not limitation to describe the features and characteristics of the present technology, to set forth the best mode of operation of the technology, and to sufficiently enable one skilled in the art to practice the technology. Accordingly, the scope of the present technology is to be defined solely by the appended claims. The following detailed description and exemplary aspects of the technology will be best understood by reference to the accompanying drawings and description, wherein the elements and features of the technology are designated by numerals throughout the drawings and described herein.

As used in this specification and the appended claims, the singular forms “a,” “an” and “the” include plural referents unless the context clearly dictates otherwise. Thus, for example, reference to “a layer” includes a plurality of such layers.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps can be performed, and certain of the stated steps can possibly be omitted and/or certain other steps not described herein can possibly be added to the method.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness can in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element can still actually contain such item as long as there is no measurable effect thereof.

As used herein, the term “about” is used to provide flexibility to a range endpoint by providing that a given value can be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 to about 80” should also be understood to provide support for the range of “50 to 80.”

An initial overview of technology is provided below and specific technology is then described in further detail. This initial summary is intended to aid readers in understanding the technology more quickly, but is not intended to identify key or essential features of the technology, nor is it intended to limit the scope of the claimed subject matter.

The technology described herein includes systems, methods, and devices for providing a determination regarding a person's health while maintaining privacy of health metrics for the person. In one example of the technology, a mobile computing device can be configured to collect one or more health metrics for a person from sensor devices in communication with the mobile computing device (e.g., sensors contained in the mobile computing device and/or in wired or wireless communication with the mobile computing device) during a baseline time period. The one or more health metrics collected by the mobile computing device can be used to define baseline health metrics and a baseline health status for a person. The mobile computing device can receive requests for a current health status for the person. In response, the mobile computing device can obtain one or more current health metrics for the person from one or more of the sensor devices in communication with the mobile computing device during a current time period. The current health metrics can be compared to the baseline health metrics to define the current health status for the person and a certification message indicating the current health status of the person can be generated at the mobile computing device. The certification message can be provided to a third-party to certify the current health status for the person.

In another example of the technology, a mobile computing device can be used by a third-party to obtain current health metrics from a person and the current health metrics can be sent to a data center to be used to define a current health status for the person. The current health status may be defined by comparing the current health metrics to baseline health metrics for the person. A certification message indicating the current health status of the person can be generated at the data center, and the certification message can be sent from the data center to the mobile computing device to allow the third-party to view the certification message.

In another example of the technology, a health check station containing a plurality of sensor devices can be used to obtain current health metrics for a person and send the current health metrics to a data center to be used to define a current health status for the person. The current health status may be defined by comparing the current health metrics to baseline health metrics for the person. A certification message indicating the current health status of the person can be generated at the data center, and the certification message can be sent from the data center to the health check station to allow a third-party to view the certification message.

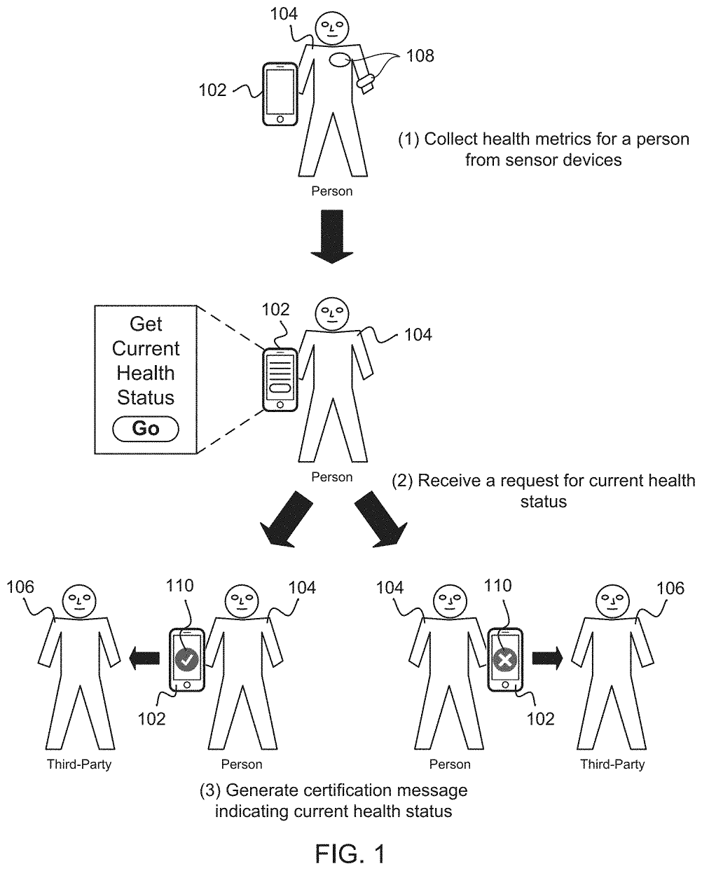

To further describe the present technology, examples are now provided with reference to the figures. FIG. 1 is a diagram illustrating a high-level example for providing a determination regarding a person's health while maintaining privacy. As illustrated, a mobile computing device 102 can be configured to provide an indication of a current health status for a person 104. The mobile computing device 102 can include a smart phone, a smart watch, a tablet, a laptop, a digital assistant, a mobile internet device, a wearable device, or another appropriate mobile computing device. The mobile computing device 102 may be kept on or in proximity of a person 102 and the mobile computing device 102 can collect one or more health metrics from sensor devices contained in the mobile computing device 102 and/or connected to the mobile computing device 102 via a wired or wireless connection.

A sensor device or system can include: a camera sensor, a heart rate sensor, a respiration rate sensor, an acoustic emission sensor, a temperature sensor, a blood pressure sensor, an EEG monitor, an ECG monitor, an blood oxygen saturation monitor, perfusion index sensor, and eye sclera color sensor, an airflow sensor, sweat-based biometric sensor, or a magnetic field monitor. In one specific aspect, the sensor device or system can include a sensor system to monitor or detect micro-visible physiological conditions and variations of an individual or subject, such as described in U.S. Pat. Nos. 9,913,583 and No. 10,470,670, which are incorporated by reference in their entirety. Essentially, the sensor system can comprise a camera in communication with a processor. The camera is configured to capture at least a first and second image of the subject. The processor comprises executable code configured to amplify microscopic temporal variations between the first and second image of the subject. The physiological variations can include, but are not limited to, changes in pulse, breathing rate, skin coloration, volume of sweat, body motions associated with a physiologic variation, and others. The processor and executable code can further be configured to generate a profile of at least one microscopic temporally detected physiological variation of the subject, and to compare the profile of the subject to a pre-existing first aggregate profile of a plurality of third-party subjects, said aggregate profile corresponding to the at least one microscopic temporally detected physiological variation of the third-party subjects, said aggregate third-party profile corresponding to a known state of the third-party subjects. The processor is further configured to detect differences between the profile of the subject and the aggregate profile of the plurality of third-party subjects and determine a probability that a state of the subject corresponds to the known state of the third-party subjects. In an alternative, the processor and executable code can be configured to compare the profile of the subject to a pre-existing baseline aggregate profile of the subject, said baseline aggregate profile corresponding to the at least one microscopic temporally detected physiological variation of the portion of the subject. In this example, the processor and executable code can be further configured to detect differences between the profile of the subject and the pre-existing baseline aggregate profile of the subject.

A sensor device can include a wearable sensor device 108 (e.g., heart rate monitor, blood pressure monitor, pulse oximeter, microphone, camera, thermometer, etc.) that connects to the mobile computing device 102 using a wired or wireless connection.

Health metrics can be generated from the sensor data received from the sensor devices. The health metrics can be securely captured and stored on the mobile computing device 102 in an encrypted form. For example, sensor data generated by a sensor device can be encrypted using an appropriate encryption technique and health metrics generated from the sensor data can be encrypted and stored on the mobile computing device 102 (or another data repository as described later). The health metrics can be collected on the computing device 102 for a baseline time period. The length of the baseline time period may be anywhere from a few hours to several days or weeks depending on the health metric. For example, a baseline time period for collecting body temperature data used to calculate a baseline body temperature may be one (1) to two (2) days or less, including at the time of the third-party health status request, whereas a baseline time period for collecting blood pressure data used to calculate a baseline blood pressure may be a week or more.

The health metrics collected by the mobile computing device 102 can be used to define baseline health metrics (e.g., health profiles) which can be compared to current health metrics obtained from the sensor devices to determine the health status of the person 104, as described in more detail below. Baseline health metrics or health profiles can be defined for individual physiological functions, such as baseline heart rate or profile, baseline blood pressure or profile, baseline respiratory rate or profile, baseline blood oxygen saturation or profile, baseline perfusion index, baseline eye sclera color, baseline microscopic temporally detected physiological variations, etc. In one example, baseline health metrics can be evaluated together to form an overall baseline health for the person 104. For example, body temperature, respiratory rate, and dehydration rate can be evaluated together to determine whether the person 104 may have symptoms associated with a defined strain of a virus. Also, the baseline health metrics can be evaluated together to determine whether a deviation in one of the baseline health metrics is normal or abnormal. For example, a rise in body temperature can be evaluated against other baseline metrics (e.g., blood pressure, oxygen saturation, etc.) to determine whether the rise in body temperature indicates a health issue or is merely an anomaly, such as increased anxiety.

In one example, in the case that baseline health metrics are not available (e.g., during a baseline time period used to collect heath metrics), public baseline health metrics can be used in place of the person's baseline health metrics. The public baseline health metrics can be an average of health metrics obtained from anonymous persons who have personal characteristics that are similar to the person 104. Illustratively, during a time that body temperature metrics are being collected to define a baseline body temperature for the person 104, physical characteristics of the person 104 (e.g., age, gender, weight, etc.) can be used to obtain a public baseline body temperature. The public baseline body temperature can be used to determine a current health status of the person 104 until a baseline body temperature has been defined for the person 104.

After one or more baseline health metrics or profiles have been defined for the person 104, the mobile computing device 102 can use the baseline health metrics to define a current health status for the person 104. In one example, the current health status can be defined by comparing one or more current health metrics to one or more baseline health metrics. The mobile computing device 102 can generate one or more current health metrics for the person 104 from sensor data obtained from the sensor devices connected to the mobile computing device 102. The types of current health metrics generated by the mobile computing device 102 may correspond to the types of baseline health metrics used to define the current health status for the person 104. For example, a current body temperature metric can be captured and compared to a baseline body temperature metric, a current heart rate metric can be obtained and compared to a baseline heart rate metric, a current microscopic temporally detected physiological variation metric can be obtained and compared to a baseline microscopic temporally detected physiological variation metric, and other types of current health metrics may be obtained and compared to corresponding types of baseline health metrics, as can be appreciated. In the case that the current health metrics correspond to the baseline health metrics, the current health status of the person can be defined as healthy. In the case that the current health metrics do not correspond to the baseline health metrics, the current health of the person 104 can be defined as not healthy. In one example, a defined threshold (e.g., within +/−3%, +/−5%, or +/−10% of a baseline health metric) can be used to determine whether the current health metrics correspond to the baseline health metrics. If the current health metrics are within the defined threshold(s), the person 104 may be categorized as healthy, and if the current health metrics are outside of the defined threshold(s), the person 104 may be categorized as not healthy. When evaluated together, the defined thresholds for the current health metrics can be adjusted based on the combination of current health metrics being used. For example, a defined threshold for a single current health metric (e.g., body temperature) when used alone to define a current health status of a person 104 may be higher/lower than a defined threshold for the current health metric when the current health metric is considered in combination with other current health metrics (e.g., respiration rate). As an illustration, when considered alone, a defined threshold for body temperature may be >98.6 F., but when evaluated in combination with a respiratory rate, the defined threshold for body temperature may be >100.4 F.

After a current health status has been defined for the person 104, a certification message 110 can be generated at the mobile computing device 102. In one aspect, the certification message 110 can be presented to a third-party 106 (e.g., third-party overseeing point of access admittance, such as transportation security or event/locale health check) to, for example, certify that the current health status of the person 104 is acceptable in association with or as part of a determination by the third-party as to whether or not to permit the person 104 to access or be admitted to a restricted or protected area, activity, locale, object etc. overseen by the third-party. As an example, prior to boarding public transportation or entering a controlled venue, the person 104 may be requested to present a certification of health indicating that the person 104 is not in danger of transmitting an infectious disease. In response, the person 104, via the mobile computing device 102, may obtain a current health status for the person 104 (i.e., themselves) and generate a certification message 110 on the mobile computing device 102 that indicates a current status of health of the person 104. The certification message 110 can be a visual indicator shown on a display screen of the mobile computing device 102. For example, the certification message 110 can be a visual icon, a visual code, a bar code, a two-dimensional (2D) bar code (e.g., QR code), an alpha-numeric code, or a color code (e.g., green or red code) shown on a display screen of the mobile computing device 102.

In one example configuration, the certification message 110 can be sent to a third-party device (not shown). The certification message 110 may be sent using RF (radio frequency) communication (e.g., BLUETOOTH, WIFI, cellular network, near-field-communication (NFC), and the like). In one example, a photograph of the person can be provided with the certification message 110 to enable the third-party 106 to verify the identity of the person 104.

In another example, a current health status for the person 104 can be presented as a defined probability of the person's health. In one example configuration, a defined probability (e.g., 82% chance of good health) can be an overall probability that the person 104 is healthy or not healthy. The defined probability can be calculated using a plurality current health metrics for the person 104 and a probability threshold that represents a baseline health for the person 104. The probability threshold may be based in part on baseline health metrics for the person 104 and a deviation from the baseline health metrics, which if exceeded, indicates an unhealthy state of the person 104. As will be appreciated, a healthy state and an unhealthy state may be defined for individual persons, where baseline health metrics or profiles for an individual person may define a healthy state, and a deviation from the baseline health metrics or profiles may be defined as an unhealthy state for the individual person. The defined probability of health can be displayed at the mobile computing device 102, which can be presented to a third-party 106, or the defined probability of health can be sent to the third-party 106 using RF communication.

In another example configuration, a defined probability can be a probability that the person 104 has a particular health condition (e.g., influenza, common cold, pneumonia, coronavirus, adenovirus, mono virus, etc.). The defined probability can be calculated using current health metrics associated with symptoms of the health condition and a probability threshold, which if not exceeded, indicates a likelihood that the person 104 has the health condition. For example, a health condition profile can be created for a particular health condition (e.g., influenza, common cold, pneumonia, coronavirus, etc.), and current health metrics for the person 104 can be compared to the health condition profile to determine a defined probability that the person 104 has the health condition. The health condition profile may specify health metrics associated with symptoms of the health condition and provide the probability threshold(s) for the health condition. The health condition profile can be provided to the mobile computing device 102 and in response to a request, the mobile computing device 102 may obtain current health metrics specified by the health condition profile and calculate a defined probability whether the person has the health condition. For example, if a defined number of the current health metrics (e.g., a majority) are below the probability threshold, the defined probability may indicate that the person 104 likely has the particular condition (e.g., influenza, common cold, pneumonia, coronavirus, etc.). The defined probability can be displayed at the mobile computing device 102 to indicate whether the person 104 likely has the particular condition, and/or the defined probability can be sent to a third-party 106 to provide an indication whether the person 104 likely has the particular condition.

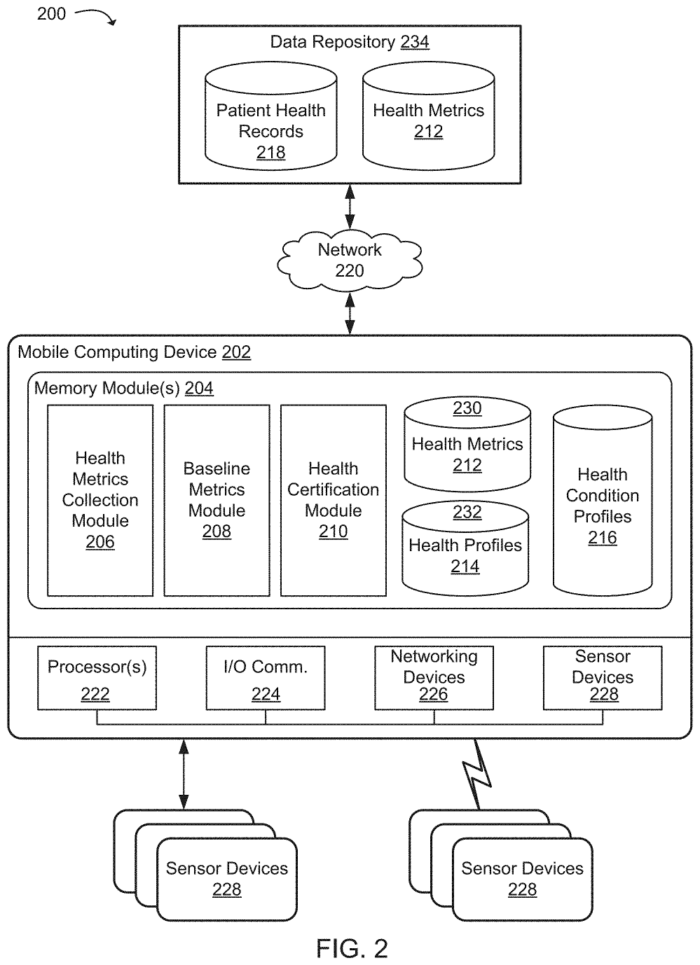

FIG. 2 is a block diagram illustrating an example system environment 200 in which the present technology may be executed. The system environment 200 may include a mobile computing device 202 that is in communication with one or more sensor devices 228. The mobile computing device 202 may include any mobile device capable of obtaining sensor data from sensor devices 228 and calculating health metrics using the sensor data. The mobile computing device 202 may be a smart phone, a smart watch, a digital assistant, a mobile internet device, a tablet computer, a laptop computer, or other mobile devices with like capability. In one example configuration, the mobile computing device 202 may be a wearable device, such as a smartwatch, medical device, smart eyewear, smart clothing, and the like containing sensor devices 228 configured to generate sensor data used to generate health metrics.

Sensor devices 228 may be contained in the mobile computing device 202 and/or may be in wired or wireless communication with the mobile computing device 202. The sensor devices 228 can include, but are not limited to: a camera sensor, a heart rate sensor, a respiration rate sensor, an acoustic emission sensor, a temperature sensor, a blood pressure sensor, an EEG sensor, an ECG sensor, an blood oxygen saturation sensor, a perfusion index sensor, an eye sclera color sensor, an airflow sensor, a sweat-based biometric sensor, a magnetic field sensor, a microscopic temporal physiological variation sensing system, and other sensor devices/systems. The sensor devices 228 can be configured to capture sensor data, from which health metrics can be calculated. For example, image data from a earners sensor can be used to generate skin pigmentation metrics, eye sclera color metrics, or microscopic temporal physiological variation-derived metrics, ECG data can be used to generate electrical heart activity metrics, EEG data can be used to generate electrical brain activity metrics, acoustics emission data can be used to generate respiratory sound metrics, pulse oximeter data can be used to generate blood oxygen saturation metrics, respiration rate metrics, and perfusion index metrics, temperature data can be used to generate body temperature metrics, and sweat data can be used to generate sweat loss metrics. As will be appreciated, the present technology is not limited to the types of health metrics described above. Any type of health metric that may be useful in defining a current health status of a person is within the scope of the disclosure.

The mobile computing device 202 can include one or more memory modules 204 configured to store processing modules, which may include a health metrics collection module 206, a baseline metrics module 208, a health certification module 210, and other modules. Also, the memory modules 204 may be used to store health metrics 212 in a health metrics data store 230, and store baseline health profiles 214 in a health profiles data store 232.

The health metrics collection module 206 can be configured to collect health metrics 212 used to define baseline health metrics and create baseline health profiles 214. The health metrics collection module 206 may be configured to periodically obtain sensor data from a sensor device 228 and generate a health metric from the sensor data. As an example, the health metrics collection module 206 may obtain sensor data from a heart rate sensor and generate a heart rate metric using the sensor data. The health metrics collection module 206 may encrypt the health metrics using an encryption technique and store the encrypted health metrics 212 to a metrics data store 230 located in the memory modules 204 of the mobile computing device 202. The health metrics 212 may include metadata describing a type of health metric and a date that the health metric 212 was created. The stored health metrics 212 may be used by the baseline metrics module 208 to calculate baseline health metrics for a person, as described below. In one example, the health metrics collection module 206 may be configured to manage health metrics 212 stored on the mobile computing device 202 by purging older health metrics 212 from the health metrics data store 230 (e.g., health metrics older than one, three, six months, etc.).

In one example, the mobile computing device 202 may be in network communication with a data repository 234 and the health metrics collection module 206 may send the health metrics 212 to the data repository 216 to allow the health metrics 212 to be stored at the data repository 234. A data repository 234 may be a cloud repository, a health care provider repository, a service provider environment, a data warehouse, an at-home computing device, or another type of data repository.

The baseline metrics module 208 can be configured to define baseline health metrics using health metrics 212 stored in the health metrics data store 230. The baseline metrics module 208 may define baseline health metrics for physiological states, such as: body temperature, heart rate, blood pressure, respiratory rate, blood oxygen saturation, eye sclera color, perfusion index, microscopic temporal physiological variation detected conditions that correlate with states, such as a physical condition (e.g., heart attack, infectious disease, etc.), a mental condition (e.g., aggravated psychosis), or an emotional condition (e.g., severe depression), and other physiological states. In one example, the baseline metrics module 208 may obtain a set of health metrics 212 from the health metrics data store 230 and calculate a baseline health metric using the set of health metrics 212. The baseline health metric may be an average of health metric values included in the set of health metrics. For example, the baseline metrics module 208 may calculate a baseline average by totaling the values of the health metric entries in the set of health metrics and dividing the sum by the number of entries in the set of health metrics.

In one aspect, weights can be assigned to deviations from baseline health metrics, where an assigned weight can reflect a dependability of having a deviation in a particular current health metric, and the weights can be summed to define a current health status for a person. For example, when used together to define a current health status, a deviation from a body temperature baseline may be weighted more or less than a deviation from a respiration baseline. In one configuration, the weighting can be dynamic. As an example, deviations in current health metrics which are indicatively linked to a health condition may be evaluated together to dynamically increase or decrease a weighting based on the relationship of the current health metrics to the health condition. For example, when both body temperature and respiration rate deviate a certain amount from baseline health metrics, the deviation when considered together may be more indicative of a health condition as compared to the individual deviations of the heath metrics, and therefore, the weighting(s) assigned to the health metrics may be increased or decreased accordingly.

In another example, the baseline metrics module 208 can be configured to create health profiles 214 for a person. A health profile 214 may be based on a plot of health metrics spaced over a time period depicting a physiological state of a person during the time period. As an illustration, a pulmonary profile for a person may be based on respiration metrics and acoustic emission metrics collected over a time period. The pulmonary profile may provide a higher-level overview of a pulmonary state for a person based on combined respiration rate metrics and respiratory sound metrics. The health profiles 214 can be stored in a health profile data store 232. In one example, the health certification module 210 can be configured to define a current health status for a person and generate a certification message that certifies the current health of the person to a third-party. The health certification module 210 may receive requests for a current health status for a person. The person may be a user or owner of the mobile computing device 202 and the person may request the current health status via a user interface for the mobile computing device 202. For example, the mobile computing device 202 may include a touchscreen and a graphical user interface displayed on the touchscreen may allow the person to select a graphical control provided to request the current health status.

In response to a request for a current health status of the person, the health certification module 210 may obtain current health metrics for the person from one or more of the sensor devices 228 contained in, and/or connected to, the mobile computing device 202. The health certification module 210 may use the current health metrics to define a current health status for the person. For example, the health certification module 210 can compare the current health metrics to a baseline health state for the person and determine whether the current health metrics correspond to the baseline health state. The baseline health state of a person may be based on one or more baseline health metrics 212, health profiles 214, and/or patient health records 218 associated with the person. Illustratively, the health certification module 210 may define a current health status of a person as healthy when one or more of the current health metrics correspond to a baseline health state of the person, and define the current health status of the person as unhealthy when one or more of the current health metrics deviate from the baseline health state of the person. For example, a current health status of a person may be defined as healthy when current health metrics for the person are substantially the same as corresponding baseline health metrics for the person (e.g., the current health metrics are within a defined threshold or range e.g., within +/−2%, +/−5%, or +/−7% of the baseline health metrics). The current health status of the person may be defined as unhealthy when the current health metrics for the person are not substantially the same as the baseline health metrics for the person (e.g., outside of the defined threshold or range of the baseline health metrics). As described earlier, when evaluated together, defined thresholds for current health metrics can be adjusted based on the combination of health metrics being used to define the current health of the person, and deviations of current health metrics front baseline health metrics can be weighted.

The health certification module 210 can be configured to generate a certification message indicating the defined current health status of a person. In one example, the health certification module 210 can output the certification message as a visual indicator and an operating system executed on the mobile computing device 202 may render the certification message on a display screen of the mobile computing device 202 which can be shown to a third-party. Illustratively, the certification message can be presented as a visual code, a bar code, a 2D bar code, an alpha-numeric code, or a color code on the display screen of the mobile computing device 202. In another example, the health certification module 210 can be configured to send the certification message (e.g., wired or wirelessly) to another device to allow a third-party to view the certification message.

In another example, the health certification module 210 can be configured to define a probability of health of a person and generate a message indicating the probability of health, which can be provided to a third-party. The defined probability can be an overall probability that the person is healthy or not healthy. The health certification module 210 may receive requests for a defined probability of health for a person, who may be a user or owner of the mobile computing device 202. In response to a request for a defined probability of health for a person, the health certification module 210 may obtain current health metrics from one or more of the sensor devices 228 contained in, and/or connected to, the mobile computing device 202. The health certification module 210 may use the current health metrics and a probability threshold or probability thresholds related to a baseline health of the person to calculate the defined probability of health of the person. The probability threshold used to calculate the defined probability of health can be based in part on baseline health metrics for the person and a deviation from the baseline health metrics (e.g., 90^{th}, 80^{th}, 70^{th}, percentile deviation), which if exceeded, indicates an unhealthy state of the person. In one example, the probability thresholds can be adjusted based on a combination of current health metrics being used to define a current health of the person, and deviations of the current health metrics from the baseline health metrics can be weighted as described earlier. As described earlier, a defined probability in one example can be a probability that a person has a particular health condition (e.g., influenza, common cold, pneumonia, coronavirus, etc.). The health certification module 210 can be configured to calculate a defined probability for a health condition using current health metrics associated with symptoms of the health condition and a probability threshold for the condition, which if exceeded, indicates a likelihood that the person has the health condition. For example, a health condition profile 216 (e.g., influenza profile, pneumonia profile, coronavirus profile, etc.) can be created for a particular health condition, and the health condition profile 216 can be used to determine a defined probability of the health condition. Health condition profiles 216 can be stored in a health profile data store 236 located on the mobile computing device 202 or in another location (e.g., data repository 234). A health condition profile 216 may specify health metrics 212 associated with symptoms of a particular health condition and specify a probability threshold for the health condition (e.g., one or more health metric thresholds or ranges associated with health condition symptoms) that, if exceeded, indicates a likelihood that a person has the health condition. For example, a health condition profile 216 may define influenza symptoms (e.g., body temperature and cough) and probability thresholds for the flu symptoms (body temperature >100° and detected cough >2 per hour). If current health metrics for a person exceed the probability thresholds, a determination can be made that the person has the influenza.

In response to a request for a defined probability of a health condition for a person, the health certification module 210 may obtain a health condition profile 216 associated with the health condition and obtain current health metrics specified by the health condition profile 216. The health certification module 210 can then calculate a defined probability whether the person has the health condition using the current health metrics using a probability threshold for the health condition specified in the health condition profile 216. The health certification module 210 can be configured to output the defined probability of the health condition to the mobile computing device 202 (e.g., as a percentage, a visual code, a bar code, a 2D bar code, an alpha-numeric code, a color code, etc.), such as for display to a third-party, or to send the defined probability of the health condition to another device to allow a third-party to view the defined probability. For example, the output may be that there is a 90% chance the owner of the device has the health condition or there is a 10% chance the owner of the device has the health condition being targeted.

The mobile computing device 202 may include hardware processor devices 222, Input/Output (I/O) communication devices 224 to enable communication between hardware devices and sensor devices 228. Networking devices 226 may be provided for communication across a network 220 with one or more remote computing devices, sensors 226, and/or data repositories 234. The networking devices 226 may provide wired or wireless networking access. Examples of wireless network access may include cellular network access, WI-FI network access, BLUETOOTH network access, or similar network access. In one example, the mobile computing device 202 can include a display, such as a touchscreen that displays an interactive graphical user interface.

The network 220 may include any useful computing network, including an intranet, the Internet, a local area network, a wide area network, a wireless data network, or any other such network or combination thereof. Components utilized for such a system may depend at least in part upon the type of network and/or environment selected. Communication over the network may be enabled by wired or wireless connections and combinations thereof. While FIG. 2 illustrates an example of a system environment used to implement the techniques above, many other similar or different environments are possible. The example system environments discussed and illustrated above are merely representative and are not meant to be limiting.

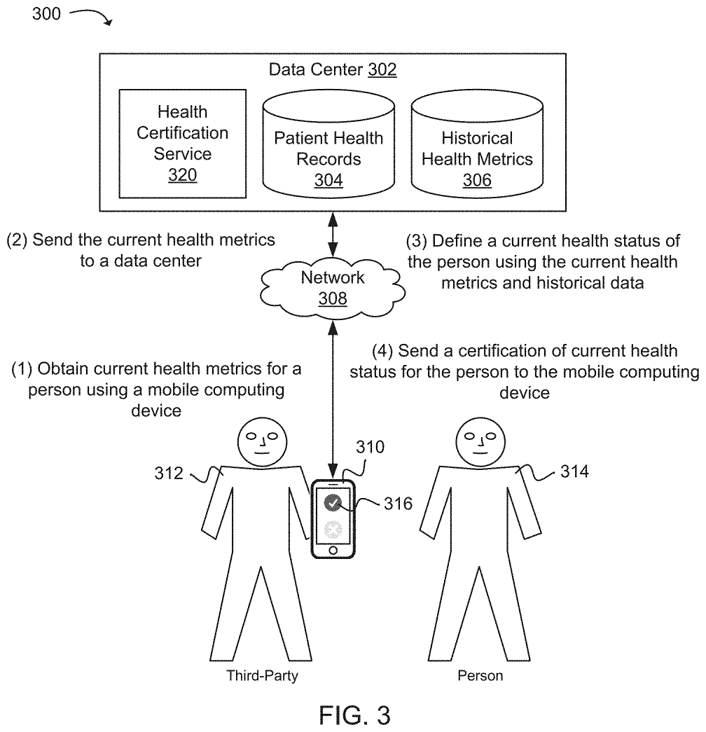

FIG. 3 is a diagram that illustrates another example system environment 300 in which the present technology may be executed. The system environment 300 can include a mobile computing device 310 used by a third-party 312 to obtain current health metrics for a person 314 and to send the current health metrics to a data center 302 where the current health metrics can be used to define a current health status for the person 314. The mobile computing device 310 can be a smart phone, a digital assistant, a smart watch, a mobile internet device, a tablet computer, a laptop computer, or other mobile devices capable of being used by a third-party to obtain current health metrics for a person 314.

In one example, the mobile computing device 310 can include one or more sensor devices and/or can be connected to one or more sensor devices via a wired or wireless connection. Sensor data received from the sensor devices can be used by the mobile computing device 310 to generate current health metrics for the person 314. The mobile computing device 310 can send the current health metrics and a personal identifier for the person 314 (e.g., personally identifiable information, bio-identifier, photograph, etc.) over a network 308 to the data center 302. The current health metrics sent to the data center 302 may be encrypted using a data encryption technique, such as asymmetric encryption. The data center 302 can be: a health care data center, cloud data center, colocation data center, managed services data center, or another type of data center. The data center 302 may host a health certification service 320 along with historical health metrics 306 and/or patient health records 304 for the person 314.

The current health metrics received at the data center 302 can be provided to the health certification service 320 hosted in the data center 302. The health certification service 320 can be configured to determine a current health status for a person 314 and generate a certification message that certifies the current health status for the person 314. In one example, the health certification service 320 identifies historical health metrics 306 and/or patient health records 304 associated with the person 314 using the personal identifier included with the current health metrics, and the health certification service 320 decrypts the current health metrics received from the mobile computing device 310 and compares the current health metrics to the historical health metrics 306 and/or patient health records associated with the person 314. As described earlier, current health metrics for a person 314 can be compared to baseline health metrics for the person 314 to define a current health status for the person 314. If the current health metrics correspond to the baseline health metrics (e.g., within a threshold), then the current health status of the person 314 can be defined as healthy. In the case that the current health metrics do not correspond to the baseline health metrics, the current health status of the person 314 can be defined as unhealthy.

After defining a current health status of the person 314, the health certification service 320 generates a certification message 316 and sends the certification message 316 to the mobile computing device 310. In response to receiving the certification message 316, the mobile computing device 310 displays the certification message 316 on a display screen to allow the third-party 312 to view the certification message 316. Accordingly, the certification message 316 can certify to the third-party 312 the current health status of the person 314. In one example, the health certification service 320 can obtain a photograph of the person 314 (e.g., from a patient health record 304) and send the photograph with the certification message 316 to the mobile computing device 310. The third-party 312 can use the photograph to verify the identity of the person 314. Also, in one example, biometrics (e.g., facial recognition, fingerprint recognition, and the like) can be used to verify the identity of the person 314. The health certification service 320 can obtain biometrics from a sensor (e.g., camera, fingerprint reader, etc.) and compare the biometrics with biometric data for the person 314 obtained from a certified file and/or access remotely-held identification data to create a form of two-factor ID certification. In one example, a blockchain technique can be used to store and obtain personally identifying information for the person 314 which can be used to verify the identity of the person 314.

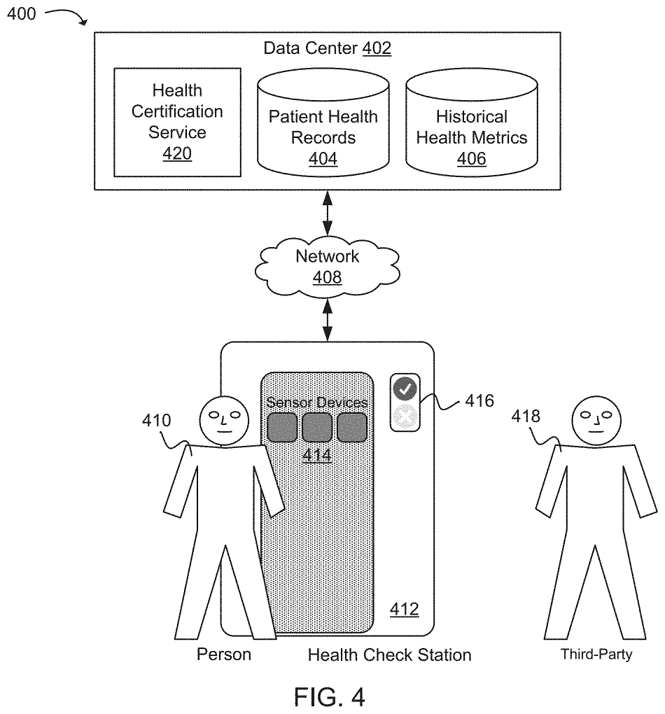

FIG. 4 is a diagram that illustrates yet another example system environment 400 in which the present technology may be executed. The system environment 400 can include a health check station 412 or kiosk containing sensor devices 414 and computing resources (e.g., a processor, computer memory, and network devices) which can be used to obtain current health metrics for a person 410 and send the current health metrics to a data center 402. Sensor devices/systems 414 included in the health check station 412 can include: a camera sensor, a heart rate sensor, a respiration rate sensor, an acoustic emission sensor, a temperature sensor, a blood pressure sensor, an EEG monitor, an ECG monitor, a blood oxygen saturation monitor, a perfusion index sensor, an airflow sensor, sweat-based biometric sensor, a magnetic field monitor, a microscopic temporal physiological detection system and other types of sensor devices. The computing resources included in the health check station 412 can be used to generate current health metrics from sensor data obtained from the sensor devices 414.

In one example, a person 410 can step up to, or into, the health check station 412 and one or more sensor devices 414 (e.g., an infrared thermometer, a pulse oximeter, a blood pressure monitor, and/or a camera device) can be used to obtain current health metrics for the person 410 (e.g., body temperature, blood oxygen saturation, blood pressure, skin pigmentation). The health check station 412 can encrypt the current health metrics and send the encrypted current health metrics to a data center 402 that hosts a health certification service 420. The health certification service 420, as described above in association with FIG. 3, can be configured to define a current health status for a person 410 by comparing the current health metrics (e.g., body temperature, blood oxygen saturation, blood pressure, skin pigmentation) to historical health metrics 406 and/or patient health records 404 (e.g., baseline body temperature, baseline blood oxygen saturation, baseline blood pressure, baseline skin pigmentation, diagnosed health condition (e.g., high-blood pressure, diabetes, etc.)). If the current health metrics correspond to baseline health metrics and/or health profiles for the person 410, then the current health status of the person 410 can be defined as healthy. In the case that the current health metrics do not correspond to the baseline health metrics and/or health profiles, the current health status of the person 410 can be defined as unhealthy.

After defining the current health status for the person 410, the health certification service 420 can generate a certification message which certifies the current health status for the person 410. In one example configuration, the health certification service 420 can send the certification message over the network 408 to the health check station 412. The health check station 412 can include a display 416 (e.g., a display screen) on which the certification message can be displayed, allowing a third-party 418 to view the certification message. In another example configuration, the health certification service 420 can send the certification message over the network 408 to a third-party device (e.g., mobile computing device, server, workstation, etc.) located at a remote location to allow a third-party to remotely view the certification message. In another example, the certification message can be sent to the person's smart phone, smart watch, tablet, laptop, digital assistant, mobile internet device, or wearable device, which could then be presented to the third party 418. In yet another example configuration, the health check station 412 can act as a physical gateway to a restricted area (e.g., an airport terminal), and the health check station 412 may allow access to the restricted area when a certification message received from the health certification service indicates that a current health status for a person 410 is healthy. Also, the health certification service 420 can be configured to verify a person's identity using any of the identification techniques described earlier.

The various processes and/or other functionality described in association with FIGS. 3-4 may be executed on one or more processors that are in communication with one or more memory modules. The data center depicted in FIGS. 3-4 may include a number of computing devices that are arranged, for example, in one or more server banks or computer banks or other arrangements. The computing devices may support a computing environment using hypervisors, virtual machine monitors (VMMs) and other virtualization software. The term “data store” may refer to any device or combination of devices capable of storing, accessing, organizing and/or retrieving data, which may include any combination and number of data servers, relational databases, object oriented databases, cluster storage systems, data storage devices, data warehouses, flat files and data storage configuration in any centralized, distributed, or clustered environment. The storage system components of a data store may include storage systems such as a SAN (Storage Area Network), cloud storage network, volatile or non-volatile RAM, optical media, or hard-drive type media. The data store may be representative of a plurality of data stores as can be appreciated.

API calls, procedure calls or other network commands that may be made in relation to the health certification service included in the data center depicted in FIGS. 3-4 may be implemented according to different technologies, including, but not limited to, Representational state transfer (REST) technology or Simple Object Access Protocol (SOAP) technology. REST is an architectural style for distributed hypermedia systems. A RESTful API (which may also be referred to as a RESTful web service) is a web service API implemented using HTTP and REST technology. SOAP is a protocol for exchanging information in the context of Web-based services.

FIGS. 3-4 illustrate that certain processing services may be discussed in connection with this technology and these services may be implemented as processing modules. In one example configuration, a module may be considered a service with one or more processes executing on a server or other computer hardware. Such services may be centrally hosted functionality or a service application that may receive requests and provide output to other services or consumer devices. For example, modules providing services may be considered on-demand computing that are hosted in a server, virtualized service environment, grid or cluster computing system. An API may be provided for each module to enable a second module to send requests to and receive output from the first module. Such APIs may also allow third parties to interface with the module and make requests and receive output from the modules.

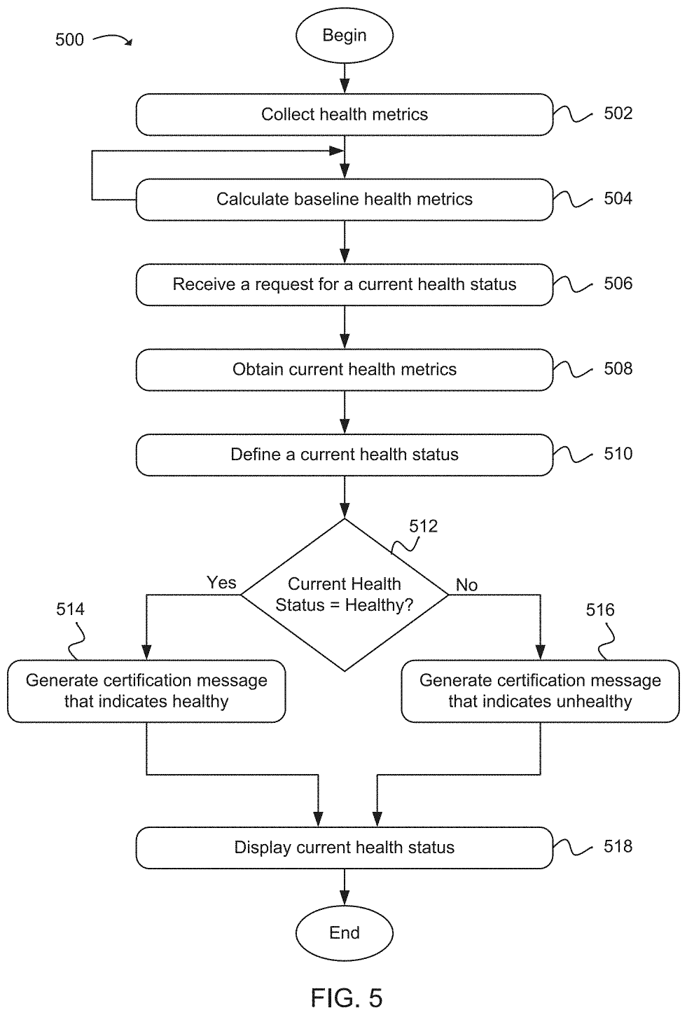

FIG. 5 is a flow diagram illustrating an example method 500 for providing a determination regarding a person's health while maintaining privacy. As in block 502, health metrics can be collected for a person. In one example configuration, a person's mobile computing device can be used to collect health metrics for the person from sensor devices contained in, and/or connected to, the mobile computing device. The health metrics can be encrypted and stored on the mobile computing device. Alternatively, the mobile computing device may encrypt the health metrics and send the encrypted health metrics to a data repository (e.g., a health care data repository, a cloud data repository, an at-home computer, etc.) where the encrypted health metrics may be stored. In another example, one or more wearable sensor devices (e.g., fitness tracker, wearable heart rate sensor, wearable thermometer, wearable blood pressure monitor, etc.) can send sensor data to a computing device (e.g., a health care server, an at-home computer, a cloud service, etc.) used to collect and store health metrics for the person. The health metrics for the person can be collected during a baseline time period (e.g., 1, 3, 10 weeks, etc.) to allow a sufficient amount of information to be gathered to calculate baseline health metrics for the person.

As in block 504, the health metrics collected by the mobile computing device can be used to define baseline health metrics for the person. The baseline health metrics can be defined for individual physiological functions, such as a baseline body temperature, a baseline heart rate, a baseline blood pressure, a baseline respiratory rate, a baseline blood oxygen saturation, a baseline perfusion index, a baseline eye sclera color, a baseline skin pigmentation, etc. In one example, baseline health metrics can be evaluated together to form an overall baseline health profile for the person. In one example, one or more baseline health profiles can be created for a person. A baseline health profile can be based on a plot of health metrics spaced over a time period. The baseline health profile can provide a physiological state of the person during the time period. The baseline health profile can be encrypted and stored on the mobile computing device or in a health profile data store located in a data repository.

As in block 506, a request for a current health status can be received at a mobile computing device via a user interface of the mobile computing device (or alternatively at a health check station as shown in FIG. 4). In response to the request, the mobile computing device can obtain one or more current health metrics that correspond to the baseline health metrics defined for the person, as in block 508, For example, the mobile computing device (or health check station) can generate current health metrics using sensor data obtained from the sensor devices contained in, and/or connected to, the mobile computing device. As in block 510, a current health status for the person can be determined using the current health metrics and baseline health metrics associated with the person. In one example configuration, one or more health profiles associated with the person can also be used to define the current health status for the person. The current health status for the person can be determined at the mobile computing device (or health check station), or the current health metrics can be sent to a health certification service via a computer network and the health certification service can determine the current health status for the person using the current health metrics.

The current health status for the person can be defined by comparing the current health metrics to the baseline health metrics (and/or the health profiles associated with the person). In one example, the current health status may be defined or determined using a binary value indicating that the person is either healthy or unhealthy. The current health status of the person may be set to a value indicating “healthy” when the current health metrics correspond to the baseline health metrics, and the current health status may be set to a value indicating “unhealthy” when one or more of the current health metrics deviate from the baseline health metrics. In one example, a defined threshold can be used to determine the current health status. As described earlier, when evaluated together, defined thresholds for current health metrics can be adjusted based on the combination of health metrics being used to define the current health of the person, and deviations of current health metrics from baseline health metrics can be weighted. If the current health metrics are within the defined threshold, the current health status may be set to “healthy”, and if the current health metrics deviate from the baseline health metrics, the current health status may be set to “unhealthy”.

After the current health status has been defined for the person, a certification message can be generated to certify the current health status for the person. As in block 512, in the case that the current health status of the person leads to a determination that the person is healthy, then as in block 514, a certification message can be generated to indicate that the person is healthy. In the case that the current health status of the person leads to a determination that the person is unhealthy, then as in block 516, a certification message can be generated to indicate that the person is unhealthy. In one example configuration, the certification message can be generated at the mobile computing device (or at a health check station) and the certification message can be displayed on the mobile computing device (or health check station), as in block 518. In another example configuration, the certification message can be generated by a health certification service which sends the certification message to the mobile computing device (or health check station) over a computer network, and the certification message can be displayed on the mobile computing device (or health check station), as in block 518.

The certification message can be visual indicator such as a visual icon, a visual code, a bar code, a 2D bar code, an alpha-numeric code, or a color code. The certification message can be viewed by the person, such that the person can be aware of the person's current health status and whether it is advisable for the person to enter a public space. Also, the certification message can be provided to a third-person (e.g., via a display screen of the mobile computing device or health check station or via sending the certification message electronically to a third-person computing device) to allow the third-person to view the certification message certifying the current health status for the person.

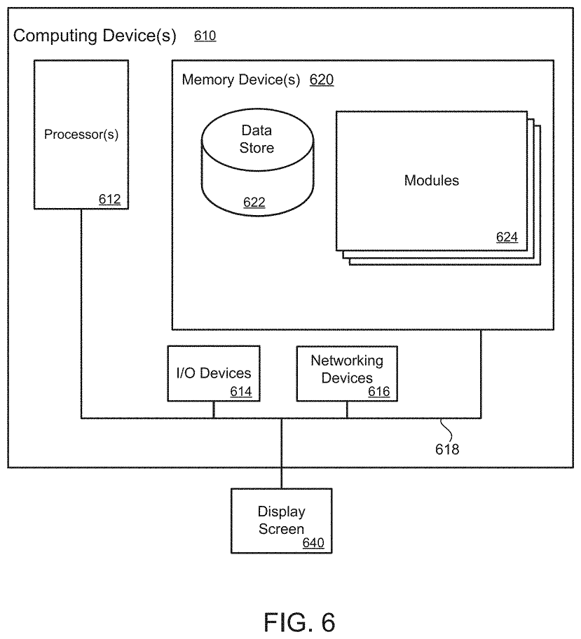

FIG. 6 illustrates a computing device 610 on which modules of this technology may execute. A computing device 610 is illustrated on which a high-level example of the technology may be executed. The computing device 610 may include one or more processors 612 that are in communication with memory devices 620. The computing device 610 may include a local communication interface 618 for the components in the computing device 610. For example, the local communication interface 618 may be a local data bus and/or any related address or control busses as may be desired.

The memory device 620 may contain modules 624 that are executable by the processor(s) 612 and data for the modules 624. In one example, the memory device 620 may include a health metrics collection module, a baseline metrics module, a health certification module, and other modules. The modules 624 may execute the functions described earlier. A data store 622 may also be located in the memory device 620 for storing data related to the modules 624 and other applications along with an operating system that is executable by the processor(s) 612.

Other applications may also be stored in the memory device 620 and may be executable by the processor(s) 612. Components or modules discussed in this description may be implemented in the form of software using high-level programming languages that are compiled, interpreted or executed using a hybrid of the methods.

The computing device 610 may also have access to I/O (input/output) devices 614 that are usable by the computing device 610. Networking devices 616 and similar communication devices may be included in the computing device 610. The networking devices 616 may be wired or wireless networking devices that connect to the internet, a LAN, WAN, or other computing network.

The components or modules that are shown as being stored in the memory device 620 may be executed by the processor(s) 612. The term “executable” may mean a program file that is in a form that may be executed by a processor 612. For example, a program in a higher level language may be compiled into machine code in a format that may be loaded into a random access portion of the memory device 620 and executed by the processor 612, or source code may be loaded by another executable program and interpreted to generate instructions in a random access portion of the memory to be executed by a processor. The executable program may be stored in any portion or component of the memory device 620. For example, the memory device 620 may be random access memory (RAM), read only memory (ROM), flash memory, a solid state drive, memory card, a hard drive, optical disk, floppy disk, magnetic tape, or any other memory components.

The processor 612 may represent multiple processors and the memory device 620 may represent multiple memory units that operate in parallel to the processing circuits. This may provide parallel processing channels for the processes and data in the system. The local communication interface 618 may be used as a network to facilitate communication between any of the multiple processors and multiple memories. The local communication interface 618 may use additional systems designed for coordinating communication such as load balancing, bulk data transfer and similar systems.

While the flowcharts presented for this technology may imply a. specific order of execution, the order of execution may differ from what is illustrated. For example, the order of two more blocks may be rearranged relative to the order shown. Further, two or more blocks shown in succession may be executed in parallel or with partial parallelization. In sonic configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions and may even be distributed over several different code segments, among different programs and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, a non-transitory machine readable storage medium, such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A method for providing a determination regarding a person's health while maintaining privacy, comprising:
collecting, at a mobile computing device, one or more health metrics for a person from sensor devices in communication with the mobile computing device during a baseline time period;
defining baseline health metrics and a baseline health status using the one or more health metrics;
receiving a request for a current health status for the person;
obtaining one or more current health metrics for the person from one or more of the sensor devices in communication with the mobile computing device, wherein the current health metrics are obtained during a current time period;
comparing the current health metrics to the baseline health metrics to define the current health status for the person; and
generating a certification message at the mobile computing device to be provided in certifying the current health status for the person.

2. The method as in claim 1, further comprising determining a person is unhealthy when one or more of the current health metrics deviate from the baseline health metrics by a defined threshold.

3. The method as in claim 1, further comprising determining the current health status for the person when the current health metrics as compared to the baseline health metrics result in a defined probability for health.

4. The method as in claim 3, further comprising determining that the person is healthy if the defined probability exceeds a probability threshold or that the person is not healthy if the defined probably is below a probability threshold.

5. The method as in claim 1, further comprising adjusting a probability threshold for a current health metric based on a combination of current health metrics used to define the current health status for the person.

6. The method as in claim 1, further comprising assigning a weight to a deviation of a current health metric from a baseline health metric based on an expected deviation, wherein weights assigned to deviations of the current health metrics are summed to define the current health status for the person.

7. The method as in claim 1, further comprising:
identifying current health metrics which are indicatively linked to a health condition; and
dynamically assigning weights to deviations of the health metrics from the baseline health metrics based on a relationship of the health metrics to the health condition.

8. The method as in claim 1, further comprising securely capturing the health metrics and storing the health metrics in an encrypted form.

9. The method as in claim 1, further comprising storing the baseline health metrics in a data store located on at least one of: the mobile computing device, an at-home computing device, a cloud repository, a health care provider repository, or a service provider environment.

10. The method as in claim 1, further comprising presenting the certification message as a visual indicator on the mobile computing device.

11. The method as in claim 1, further comprising, verifying an identity of the person using biometrics obtained from a sensor device included in the mobile computing device.

12. The method as in claim 1, further comprising storing personally identifying information for the person in a blockchain; and
retrieving the personally identifying information from the blockchain to verify an identity of the person.

13. The method as in claim 7, further comprising presenting the certification message as at least one of: a visual code, a bar code, a 2D bar code, an alpha-numeric code, or a color code on a display screen of the mobile computing device.

14. The method as in claim 1, further comprising sending the certification message to a third-party device via an RF (radio frequency) communication.

15. The method as in claim 1, wherein the sensor devices are in the mobile computing device or are electrically networked with the mobile computing device.

16. The method as in claim 1, wherein the sensor devices comprise at least one of: a heart rate sensor, a respiration rate sensor, a quality of respiration sensor, a respiration profile sensor, an acoustic emission sensor, a temperature monitor, a skin color monitor, a skin color pattern monitor, blood pressure sensor, a blood pressure profile sensor, an EEG monitor, an ECG monitor, an blood oxygen saturation monitor, a perfusion index sensor, an eye sclera color sensor, an airflow sensor, a microscopic temporal physiological variation sensing system, or a magnetic field monitor.

17. The method as in claim 1, further comprising generating a health metric profile using sensor data generated by a sensor device.

18. The method as in claim 12. wherein the health metric profile includes at least one of a blood pressure profile created using blood pressure metrics obtained from a blood pressure sensor, a pulmonary profile created using pulmonary metrics obtained from a pulmonary sensor, a heart rate profile created using heart rate metrics obtained from a heart rate sensor, or a microscopic temporal physiological variation profile using microscopic temporal physiological variation metrics.

19. The method as in claim 1, wherein the mobile computing device is a smart phone, smart watch, tablet, laptop, a digital assistant, a mobile internet device, or a wearable device.

20. A mobile computing device to provide a determination regarding a person's health while maintaining privacy, comprising:
at least one processor;
a plurality of sensor devices to monitor a person's medical metrics, wherein the plurality of sensor devices are in communication with the mobile computing device;
at least one memory device including a data store to store a plurality of data and instructions that, when executed, cause the mobile computing device to:
collect one or more health metrics for a person from the plurality of sensor devices in communication with the mobile computing device during a baseline time period;
define baseline health metrics and a baseline health status using the one or more health metrics;
receive a request for a current health status for the person;
receive current health metrics for the person from the plurality of sensor devices in communication with the mobile computing device, wherein the current health metrics are obtained during a current time period;
compare the current health metrics to the baseline health metrics to define a current health status for the person; and
display a certification message certifying the person's current health status on a screen of the mobile computing device to enable the certification message to be provided to a third-party.

21. The mobile computing device as in claim 20, wherein the instructions of the at least one memory device further cause the mobile computing device to securely capture the health metrics and store the health metrics in an encrypted form.

22. The mobile computing device as in claim 20, wherein the instructions of the at least one memory device further cause the mobile computing device to present the certification message as a visual icon on the mobile computing device.

23. The mobile computing device as in claim 20, wherein the instructions of the at least one memory device further cause the mobile computing device to present the certification message as at least one of: a visual code, a bar code, a 2D bar code, an alpha-numeric code, or a color code on a display screen of the mobile computing device.

24. The mobile computing device as in claim 20, wherein the instruction of the at least one memory device further cause the mobile computing device to present a picture of the person with the certification message.

25. The mobile computing device as in claim 20, wherein the plurality of sensor devices comprise at least one of: a heart rate sensor, a quality of heart rate sensor, a respiration rate sensor, a quality of respiration sensor, a respiration profile sensor, an acoustic emission sensor, a temperature monitor, a skin color monitor, a skin color pattern monitor, blood pressure sensor, a blood pressure profile sensor, EEG monitor, ECG monitor, blood oxygen saturation monitor, a perfusion index sensor, an eye sclera color sensor, an airflow sensor, a microscopic temporal physiological variation sensing system, or a magnetic field monitor.

26. A non-transitory machine readable storage medium having instructions embodied thereon, the instructions when executed by one or more processors, cause the one or more processors to perform a process, comprising:
receiving one or more health metrics for a person from a plurality of sensor devices in communication with a mobile computing device during a baseline time period;
defining baseline health metrics and a baseline health status using the one or more health metrics;
receiving a request for a current health status for the person;
receiving current health metrics for the person from the plurality of sensor devices in communication with the mobile computing device, wherein the current health metrics are obtained during a current time period;
comparing the current health metrics to the baseline health metrics to define a current health status for the person; and
displaying a certification message certifying the current health status of the person on a screen of the mobile computing device to enable the certification message to be provided to a third-party.

27. The non-transitory machine readable storage medium as in claim 26, the process further comprising displaying the certification message as at least one of: a visual code, a bar code, a 2D bar code, an alpha-numeric code, or a color code on a display screen of a mobile computing device.

28. The non-transitory machine readable storage medium as in claim 26, wherein the plurality of sensor devices comprise at least one of: a heart rate sensor, a quality of heart rate sensor, a respiration rate sensor, a quality of respiration sensor, a respiration profile sensor, an acoustic emission sensor, a temperature monitor, a skin color monitor, a skin color pattern monitor, blood pressure sensor, a blood pressure profile sensor, EEG monitor, ECG monitor, blood oxygen saturation monitor, a perfusion index sensor, an eye sclera color sensor, an airflow sensor, a microscopic temporal physiological variation sensing system, or a magnetic field monitor.
